(19) United States
(12) Patent Application Publication
(10) Pub. No.: US 2006/0121465 A1
(43) Pub. Date: Jun. 8, 2006

(54) Sgk and nedd used as diagnostic and therapeutic targets

(76) Inventors: Florian Lang, Tuebingen (DE); Karl Lang, Tuebingen (DE); Michael Dieter, Reutlingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/516,745

(22) PCT Filed: Jun. 4, 2003
(86) PCT No.: PCT/EP03/05847

§ 371 Date: Oct. 12, 2005

(30) Foreign Application Priority Data
Jun. 4, 2002 (DE) 102 25 844.9

(51) Int. Cl. C12Q 1/68; G01N 33/53

(52) U.S. Cl. 435/6; 435/7.1

(57) ABSTRACT

The invention relates to the use of a substance for the diagnostic detection of Sgk, especially Sgk1 and/or Sgk3, and/or PKB and/or Nedd, especially Nedd4-2, and to the use of active ingredients which influence glucose transport for treating diseases associated with disturbed glucose transport, and for fattening animals. The invention also relates to a diagnosis kit and a pharmaceutical composition comprising an active quantity of at least one active ingredient which influences glucose transport. The invention further relates to a method for producing transgenic animals.

The present invention relates to the use of a substance for diagnostically detecting Sgk (serum and glucocorticoid-dependent kinase), in particular Sgk1 and/or Sgk3, and/or protein kinase B (PKB) and/or Nedd (neural precursor cell-expressed developmentally down-regulated gene), in particular Nedd4-2. The invention furthermore relates to the use of an active compound for exerting an effect on glucose transport, in particular for the therapeutic treatment of diseases which are connected with disturbed glucose absorption and for increasing the weight of animals during fattening. The invention also relates to a diagnostic kit.

The Na⁺-coupled transporter Sglt1 (sodium glucose transporter) in the apical membrane of the epithelial cells is responsible for the intestinal and renal transport of glucose. A disturbance in this glucose transport can lead to a variety of diseases such as obesity and diabetes mellitus.

Thus far, little is known about the regulation of Sglt1. A novel mechanism which regulates the renal epithelial Na⁺ channel ENaC has recently been discovered: The channel is ubiquinated by the ubiquitin ligase Nedd4-2 and thereby prepared for internalizing and breakdown [Debonneville C, Flores S Y, Kamynina E, Plant P J, Tauxe C, Thomas M A, Munster C, Chraibi A, Pratt J H, Horisberger J D, Pearce D, Loffing J, Staub O. Phosphorylation of Nedd4-2 by Sgk1 regulates epithelial Na(+) channel cell surface expression. EMBO J. 2001; 20: 7052-7059]. Nedd4-2 is phosphorylated, and thereby inactivated, by the serum- and glucocorticoid-inducible kinase 1 (Sgk1). Consequently, Sgk1 is a potent stimulator of the renal epithelial Na⁺ channel [De la Rosa et al. 1999, Boehmer et al. 2000, Chen et al. 1999, Naray-Fejes-Tóth et al. 1999, Lang et al. 2000, Chigaev et al. 2000, Wagner et al. 2001].

Recently, a study of twins has shown that certain single nucleotide polymorphisms (SNPs) in the sgk1 gene (E8CC/CT;I6CC) are associated with elevated blood pressure [Busjahn A, Aydin A, Uhlmann R. et al., Serum- and glucocorticoid-regulated kinase (SGK1) gene and blood pressure. Hypertension 2002; 40:256-260].

In a general manner, kinases are proteins which transfer a phosphate group to individual substrates. The serum- and glucocorticoid-dependent kinase (Sgk) was originally cloned from rat mammary carcinoma cells [Webster M K, Goya L, Firestone G L, Y. Biol. Chem. 268 (16): 11482-11485, 1993; Webster M K, Goya L, Ge Y, Maiyar A C, Firestone G L, Mol. Cell. Biol. 13 (4): 2031-2040, 1993].

Sgk1 was originally cloned as a glucocorticoid-sensitive gene [Webster M K, Goya L, Ge Y, Maiyar A C, Firestone G L: Characterization of Sgk, a novel member of the serine/threonine protein kinase gene family which is transcriptionally induced by glucocorticoids and serum. Mol Cell Biol 1993; 13: 2031-2040]. A number of investigations have revealed that Sgk1 is under the influence of a large number of stimuli [Lang F, Cohen P. Regulation and physiological roles of serum- and glucocorticoid-induced protein kinase isoforms. Science STKE. 2001 Nov. 13; 2001 (108): RE17], such as that of the mineral corticoids [Chen S Y, Bhargava A, Mastroberardino L, Meijer O C, Wang J, Buse P, Firestone G L, Verrey F, Pearce D: Epithelial sodium channel regulated by aldosterone-induced protein Sgk. Proc Natl Acad Sci USA 1999; 96: 2514-2519; Náray-Fejes-Tóth A, Canessa C, Cleaveland E S, Aldrich G, Fejes-Tóth G: Sgk is an aldosterone-induced kinase in the renal collecting duct. Effects on epithelial Na⁺ channels. J Biol Chem 1999; 274: 16973-16978; Park J, Leong M L, Buse P, Maiyar A C, Firestone G L, Hemmings B A: Serum and gluco-corticoid-inducible kinase (Sgk) is a target of the PI 3-kinase-stimulated signaling pathway. EMBO J 1999; 18: 3024-3033; Brenan F E, Fuller P J. Rapid upregulation of serum and glucocorticoid-regulated kinase (Sgk) gene expression by corticosteroids in vivo. Mol Cell Endocrinol. 2000; 30: 166: 129-36; Cowling R T, Birnboim H C. Expression of serum- and glucocorticoid-regulated kinase (Sgk) mRNA is up-regulated by GM-CSF and other proinflammatory mediators in human granulocytes. J Leukoc Biol. 2000; 67; 240-248], inter alia. Sgk1 is stimulated by insulin-like growth factor IGF1, by insulin and oxidative stress by way of a signal cascade, and by phosphoinositol-3-kinase (PI3-kinase) and phosphoinositol-dependent kinase (Pdk1) [Kobayashi T, Cohen P. Activation of serum- and glucocorticoid-regulated protein kinase by agonists that activate phosphatidylinositide 3-kinase is mediated by 3-phosphoinositide-dependent protein kinase-1 (Pdk1) and pdk2. Biochem J 1999; 339: 319-328; Park J, Leong M L, Buse P, Maiyar A C, Firestone G L, Hemmings B A: Serum and glucocorticoid-inducible kinase (Sgk) is a target of the PI 3-kinase-stimulated signaling pathway. EMBO J 1999; 18: 3024-3033; Kobayashi T, Deak M, Morrice N, Cohen P. Characterization of the structure and regulation of two novel isoforms of serum- and gluco-corticoid-induced protein kinase. Biochem. J. 1999; 344: 189-197]. The activation of Sgk1 by Pdk1 involves a phosphorylation at the serine at position 422. Mutation of this serine into an aspartate (^{S422D}Sgk1) leads to a kinase which is constitutively active [Kobayashi T, Cohen P: Activation of serum- and glucocorticoid-regulated protein kinase by agonists that activate phosphatidylinositide 3-kinase is mediated by 3-phosphoinositide-dependent protein kinase-1 (Pdk1) and pdk2. Biochem J 1999; 339: 319-328].

Since then, two isoforms of Sgk1, i.e. Sgk2 and Sgk3, have been cloned [Kobayashi T, Deak M, Morrice N, and Cohen P. 1999. Characterization of the structure and regulation of two novel isoforms of serum- and glucocorticoid-induced protein kinase. Biochem J. 344:189-197]. All three Sgk isoforms, and protein kinase B (PKB), are activated by way of PI3 kinase and Pdk1 [Kobayashi, T., and Cohen, P. 1999. Activation of serum- and glucocorticoid-regulated protein kinase by agonists that activate phosphatidylinositide 3-kinase is mediated by 3-phosphoinositide-dependent protein kinase-1 (PDK1) and PDK2. Biochem J. 339:319-328].

The aim of the invention is to provide novel diagnostic and therapeutic applications for the regulation of glucose uptake. It is furthermore an aim of the invention to provide applications which increase the bodyweight of animals by regulating glucose uptake.

Surprisingly, it has been demonstrated, in two-electrode voltage clamp experiments, that Nedd4-2 also inactivates the renal and intestinal Na⁺ glucose transporter Sglt and that this effect is suppressed by Sgk1 and/or Sgk3 and/or PKB. Since accelerated glucose absorption promotes the development of obesity, for example, it follows that Nedd4-2, Sgk1, Sgk3 and PKB play a causal role in the development of obesity. By means of detecting Nedd4-2 and/or Sgk1 and/or Sgk3 and/or PKB, the cause of the obesity can, for example, be identified and treated or prevented by means of appropriate therapeutic and prophylactic measures. The obesity, and also the hyperglycemia, which are induced by accelerated intestinal glucose absorption also favor the development of diabetes mellitus. Finally, simultaneous dysregulation of the renal Na⁺ channels would result in the development of hypertension. Obesity, hypertension and the development of diabetes mellitus are key features of what is termed the metabolic syndrome.

Conversely, it follows that inhibition of Sgk1 and/or Sgk3 and/or PKB in turn leads to inhibition of the renal and intestinal Na⁺ glucose transporter Sglt.

Accordingly, the object according to the invention is achieved by the subject matter of the independent claims 1, 10, 13, 23, 28, 30, 31, 32, 34 and 46. Preferred embodiments are specified in the dependent claims. The wording of all the claims is hereby incorporated into the description by reference.

The invention claims the use of at least one substance for detecting the expression and/or function of activated and/or inactivate Sgk, in particular Sgk1 and/or Sgk3, and/or PKB and/or Nedd, in particular Nedd4-2. This thereby also makes it possible, in particular, to diagnose diseases which are associated with disturbed glucose transport. The substance is preferably at least one substance from the group of antibodies and/or nucleotides. For example, the substance can be an antibody which is directed against Sgk1, Sgk3, PKB and/or Nedd4-2 and can be employed in a detection method which is known to the skilled person, such as ELISA (enzyme-linked immunosorbent assay). In these immunoassays, the specific antibody (or homologous test antigens in the case of antibody determinations) which is directed against the antigen to be determined (e.g. Sgk1, Sgk3 and/or PKB) is bound to a support substance (e.g. cellulose or polystyrene) on which immune complexes are formed following incubation with the sample. In a subsequent step, these immune complexes are supplied with a labeled antibody. By means of adding a chromogenic substrate to the reaction mixture, the immune complex-bound enzyme/substrate complexes can be visualized or the antigen concentration in the sample can be ascertained by photometrically determining the immune complex-bound label enzymes by comparing with standards of known enzyme activity. As already mentioned above, it is also possible, for the diagnostic detection, to use nucleotides, in particular oligonucleotides, which are suitable for providing, for example using the polymerase chain reaction, a quantitative detection of Sgk1, for example, by means of a molecular genetic method in which particular DNA segments are amplified selectively.

Preference is given to using antibodies which are directed against at least one phosphorylated and/or unphosphorylated kinase consensus sequence in the Nedd protein. In this connection, “consensus sequence” is to be understood as meaning the amino acid sequences which form the substrate site of the kinases, that is the site(s) of the phosphorylation. The Sgk1 consensus sequence in the Nedd protein is particularly preferred in this context.

It is also possible that inactivating mutations in the Nedd protein, in particular in the kinase consensus sequence (e.g. ^{S338D}Nedd4-2 or ^{S444D}Nedd4-2) are detected. Furthermore, an activating mutation, for example ^{S422D}Sgk1 and/or ^{T308D,S473D}PKB is detected in the DNA of the patients. In a further use, corresponding mutations are detected in the RNA of the patients. Finally, corresponding mutations are detected in the Sgk, in particular Sgk1 and/or Sgk3, PKB and/or Nedd protein, in particular in the Nedd4-2 protein, of the patients. Preference is given to using either suitable antibodies and/or suitable nucleotides, in particular oligonucleotides, as probes for these detections.

The diseases which are associated with disturbed glucose transport and which are to be diagnosed are, in particular, the metabolic syndrome or obesity.

The invention furthermore encompasses a method for diagnosing predispositions for corpulence or obesity. This diagnostic method is characterized in that at least one polymorphism is detected in sgk, in particular sgk1 and/or sgk3, in a gene for PKB, nedd, in particular nedd4-2, and/or in sglt, in particular sglt1. Particular preference is given, in this connection, to detecting the E8CC/CT;I6CC polymorphism in sgk1. This polymorphism is directly correlated with the body mass index such that it is a particularly suitable marker for highlighting predispositions to corpulence. This abbreviation stands for an SNP(C→T) in Exon 8 and a second SNP (T→C) which is located at a distance of 551 base pairs from the donor site (Intron 6) of Exon 7. For the purpose of detecting corresponding polymorphisms, preference is given to removing blood from appropriate experimental animals or patients and using the genetic materials which are contained therein to determine the sequence at the corresponding site by means of appropriate sequencing or by using other methods with which the skilled person is familiar. Aside from blood, all other biological samples from which genetic material can be isolated are also in principle suitable.

The invention furthermore claims the use of at least one active compound for exerting an effect on glucose transport, in particular intestinal and/or renal glucose transport. The glucose transporter Sglt, in particular Sglt1, is preferably at least partially responsible for this glucose transport. According to the invention, the glucose transport can be affected by exerting an effect on the expression and/or activity of Sglt, in particular Sglt1. The active compound preferably exerts an effect on at least one Sgk, in particular Sgk1 and/or Sgk3, and/or PKB, and/or an effect on at least one Nedd, in particular Nedd4-2. The active compound is preferably directed against an Sgk, in particular Sgk1 and/or Sgk3, and/or PKB and/or a Nedd, in particular Nedd4-2. In another preferred embodiment of the invention, the active compound is directed against activators, inhibitors, regulators and/or biological precursors of an Sgk, in particular of Sgk1 and/or Sgk3, and/or PKB and/or a Nedd, in particular Nedd4-2.

In a preferred embodiment of the invention, the active compound is a polynucleotide. This polynucleotide can, for example, comprise an antisense sequence which decreases or inhibits the expression of at least one of said proteins. In another preferred embodiment, the polynucleotide encodes a peptide, preferably a polypeptide, with this peptide exerting an effect on the expression and/or function of an Sgk, in particular Sgk1 and/or Sgk3, and/or PKB and/or a Nedd, in particular Nedd4-2. Furthermore, the active compound can itself preferably be a peptide or a polypeptide which exerts an effect on the expression and/or function of said proteins. The active compound can be a “small molecular compound”, preferably a “small molecular compound” having a molecular weight of <1000.

Depending on whether the aim is that of treating diseases which are associated with disturbed glucose transport or whether the aim is to increase the bodyweight of animals in connection with fattening, the respective enzymes have to be affected in different ways. For the purpose of preventing or treating diseases which are connected with disturbed glucose absorption, the active compound should inhibit at least one Sgk, in particular Sgk1 and/or Sgk3, and/or PKB, and/or stimulate at least one Nedd, in particular Nedd4-2. Since Sgk and PKB are kinases, kinase inhibitors which are known to the skilled person, such as staurosporine and/or chelerythrine, or at least one of their analogs, is/are suitable, in particular. Since Nedds are ligases, ligase activators are suitable for stimulating them. These active compounds are preferably used for producing a drug or a pharmaceutical composition. The diseases which are to be treated are preferably the metabolic syndrome, in particular obesity.

If, on the other hand, in contrast to the above-described prevention or treatment of diseases in which the aim is to lower glucose transport, an increase in glucose transport, for example for the purpose of increasing the bodyweight of animals in connection with fattening, is to be achieved, the active compound preferably stimulates at least one Sgk, in particular Sgk1 and/or Sgk3, and/or PKB, and/or inhibits at least one Nedd, in particular Nedd4-2. Stimulating Sgk1, for example, results in Nedd4-2, for example, being inhibited, with this in turn leading to the breakdown of the glucose transporter Sglt1 being delayed. This in turn results in glucose transport being increased. In a preferred embodiment of the invention, the active compound is at least one Sgk activator and/or PKB activator, in particular a growth factor, preferably IGF1 and/or insulin.

In another preferred embodiment of the invention, the active compound is at least one stimulant of the transcription of sgk1 and/or sgk3 and/or a gene for PKB, preferably at least one glucocorticoid, mineral corticoid, gonadotropin and/or cytokine, in particular TGFβ.

The invention furthermore relates to a diagnostic kit. This kit comprises at least one substance for detecting the expression and/or function of activated and/or inactive Sgk, in particular Sgk1 and/or Sgk3, and/or PKB and/or Nedd, in particular Nedd4-2, for diagnosing diseases which are associated with disturbed glucose transport. The diseases are preferably the metabolic syndrome, in particular obesity. The kit can, in particular, contain antibodies and/or oligonucleotides for detecting the corresponding proteins and/or nucleic acids. For example, these antibodies and/or oligonucleotides can be used for analyzing the quantity and/or activity of the different proteins or enzymes. It is furthermore also possible to detect corresponding mutations in the genes. The reader is referred to the remaining description with regard to additional features of this kit.

In addition to this, the invention encompasses antibodies which are directed against at least one phosphorylated kinase consensus sequence in a Nedd protein. This kinase consensus sequence is the sequence which is phosphorylated by a corresponding kinase, in particular by Sgk1. The antibody preferably recognizes the kinase consensus sequence in the Nedd4-2 protein. Using such an antibody it is possible to analyze whether Nedd4-2 was phosphorylated by Sgk1 and thereby inactivated. This therefore consequently makes it possible to investigate the activity status of Nedd4-2. The invention further comprises an antibody which is directed against the corresponding unphosphorylated kinase consensus sequence in the Nedd protein. Particular preference is given to combining the two antibodies according to the invention in one test setup, with this making it possible to obtain very informative results with regard to the activity status of Nedd.

The invention also comprises antibodies which are directed against at least one mutated kinase consensus sequence in a Nedd protein. This consensus sequence is in turn preferably the Sgk1 consensus sequence which is mutated correspondingly. The kinase consensus sequence is preferably located in the Nedd4-2 protein. Mutants which are particularly preferred in this connection are ^{S338D}Nedd4-2 and/or ^{S444D}Nedd4-2. The effect of corresponding mutations is that Nedd can no longer be phosphorylated by a corresponding kinase, in particular Sgk1. Such an antibody can be used as a helpful tool for investigating corresponding mutants.

The antibodies according to the invention are prepared using methods which are familiar to the skilled person. In particular, it is possible to prepare polyclonal or monoclonal antibodies, with monoclonal antibodies being preferred because of what is in general their higher specificity.

The described antibodies can particularly advantageously be used in the diagnostic kit according to the invention. Furthermore, the described antibodies can also very advantageously be employed in the use according to the invention for detecting the expression and/or function of Sgk, PKB and/or Nedd. In this context, the antibodies can be used in accordance with customary immunological methods. In particular, it is possible to use these antibodies to carry out the ELISAs which have already been mentioned.

The invention additionally encompasses a composition, preferably a pharmaceutical composition, which comprises at least one active compound which exerts an effect on glucose transport, in particular intestinal and/or renal glucose transport, and, where appropriate, a pharmaceutically acceptable excipient. Particularly preferably, the active compound exerts an effect on at least one Sgk and/or PKB and/or at least one Nedd. In another preferred embodiment, the active compound exerts an effect on activators, inhibitors, regulators and/or biological precursors of an Sgk, in particular of Sgk1 and/or Sgk3, and/or PKB and/or a Nedd, in particular Nedd4-2.

The active compound is advantageously a poly-nucleotide. This polynucleotide can comprise or form an antisense sequence which reduces or inhibits the expression of the corresponding genes. It is furthermore possible to select a corresponding polynucleotide such that it inhibits the expression of the respective gene or genes by means of a dominant negative approach, as known to the skilled person, or limits the function of the corresponding gene products. Furthermore, the polynucleotide can encode a peptide, preferably a polypeptide, with this peptide exerting an effect on the expression and/or function of an Sgk, in particular Sgk1 and/or Sgk3, and/or PKB and/or a Nedd, in particular Nedd4-2. The corresponding molecular biological procedures which are required for these approaches are accessible to the skilled person. In another preferred embodiment, the active compound is the described peptide itself. The active compound is preferably a “small molecular compound”, preferably a “small molecular compound” having a molecular weight of <1000.

Particularly for the purpose of treating diseases which are associated with disturbed glucose transport, the active compound inhibits at least one Sgk and/or PKB and/or stimulates at least one Nedd. For treating these diseases, the active compound is particularly preferably at least one kinase inhibitor, preferably staurosporine and/or chelerythrine or one of their analogs, and/or at least one ligase activator.

For the purpose of increasing glucose transport, in particular in the connection with animal fattening, the active compound preferably stimulates at least one Sgk and/or PKB and/or inhibits at least one Nedd. For increasing glucose transport, the active compound is advantageously an Sgk1 activator, in particular a growth factor, preferably IGF1, and/or insulin. In another preferred embodiment of the invention, the active compound is a stimulant of the transcription of Sgk1 and/or Sgk3 and/or PKB, preferably at least one glucocorticoid, mineral corticoid, gonadotropin and/or cytokine, in particular TGFβ.

The different possibilities which have been described can also be combined with each other.

The invention furthermore encompasses a method for producing transgenic animals which exhibit an increase in lipid deposition in adipose tissue. Humans are excluded from this aspect of the invention. These animals are of great interest for food production, in particular, since they put on weight more rapidly. Fattening can be carried out much more rapidly and more efficiently using these animals. The method for producing these animals is characterized in that the expression and/or function of Sglt, in particular Sglt1, is increased in these animals. This thereby accelerates the intestinal absorption of glucose, with this leading to a more rapid increase in the glucose concentration in the plasma. This results in higher levels of insulin being secreted, with this finally leading to lipid deposition in adipose tissue being stimulated.

In a particularly preferred embodiment of this aspect of the invention, sglt, in particular sglt1, is, for this purpose, overexpressed in the animal. This is effected, for example, by introducing appropriate gene constructs, in particular vectors, which carry appropriately strong promoters which are functionally located upstream of an appropriate sglt sequence. Preference is also given to cloning animals which exhibit appropriately strong expression of sglt, in particular sglt1. The methodological procedures for doing this are accessible to the skilled person.

In another preferred embodiment, the expression and/or function of Sgk, in particular Sgk1 and/or Sgk3, and/or of PKB, is/are increased. In the final result, this thereby also increases the activity, or the protein quantity, of Sglt, in particular Sglt1, which means that glucose transport is increased. To do this, the corresponding genes can be overexpressed using customary molecular biological methods. On the other hand, gene constructs which express appropriate constitutively active mutants can also be introduced or integrated into the organism. The mutants ^{S422D}sgk1 and/or ^{T308D,S473D}PKB are particularly preferred in this connection. The activity of these mutants is independent of other activating enzymes, in particular kinases, and the mutants are therefore constantly active. They inhibit the breakdown of Sglt, in particular Sglt1, which is brought about by the ubiquitin ligase Nedd, in particular Nedd4-2, with this resulting in glucose transport being increased.

In another preferred embodiment, the expression and/or function of the ubiquitin ligase Nedd, in particular Nedd4-2, is decreased. This also has the effect of increasing glucose transport as a result of Sglt, in particular Sglt1, being broken down to a reduced extent. An appropriate reduction in the expression and/or function of Nedd can likewise be achieved using customary molecular biological methods such as antisense or dominant-negative approaches. Particular preference is given to stably integrating suitable mutations of nedd, in particular nedd4-2, into the organism or to switching off the negative gene for Nedd in order, in this way, to decrease or inhibit the expression of this enzyme over a long period. Appropriate procedures are known to the skilled person. Particular preference is given, in this connection, to inserting at least one inactivating mutation into Nedd, in particular Nedd4-2. The mutations ^{S338D}nedd4-2 and/or ^{S444D}nedd4-2 can very advantageously be used in this context. The invention likewise encompasses animals which can be produced by the method according to the invention.

The features which have been described, and other features of the invention, ensue from the following description of preferred embodiments in combination with the subclaims and the figures. In this connection, the individual features can in each case be realized on their own or with several of them being combined with each other.

In the figures:

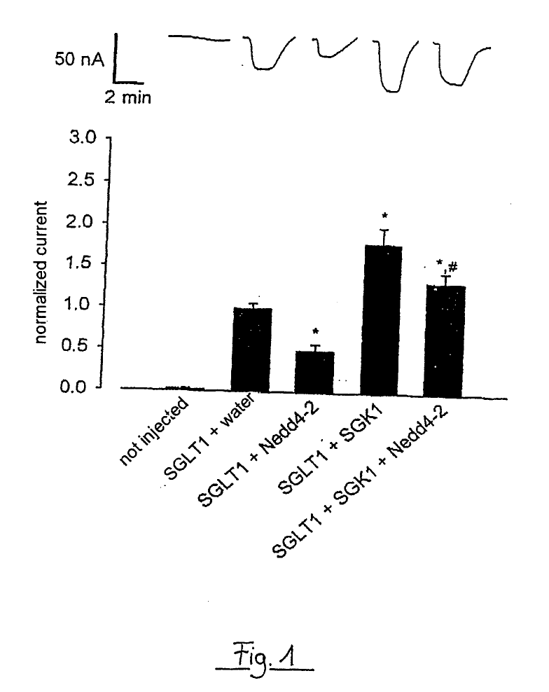

FIG. 1: shows the regulation of the Na⁺-coupled glucose transporter Sglt1 by Nedd4-2 and Sgk1.

Upper section: Originally measured data; lower section: arithmetic means±SEM (n=6-15). Xenopus laevis oocytes were injected with sglt1, nedd4-2 and/or sgk1 cRNA. Whereas Nedd4-2 downregulated the currents which are induced by 20 mM glucose which in oocytes which were expressing Sglt1, Sgk1 stimulated the currents and reversed the effect of Nedd4-2.
* indicates the significant differences as compared with the currents which were measured in oocytes which were only expressing Sglt1.
# indicates the significant differences as compared with the corresponding values in oocytes which were expressing Sglt1 and Nedd4-2.

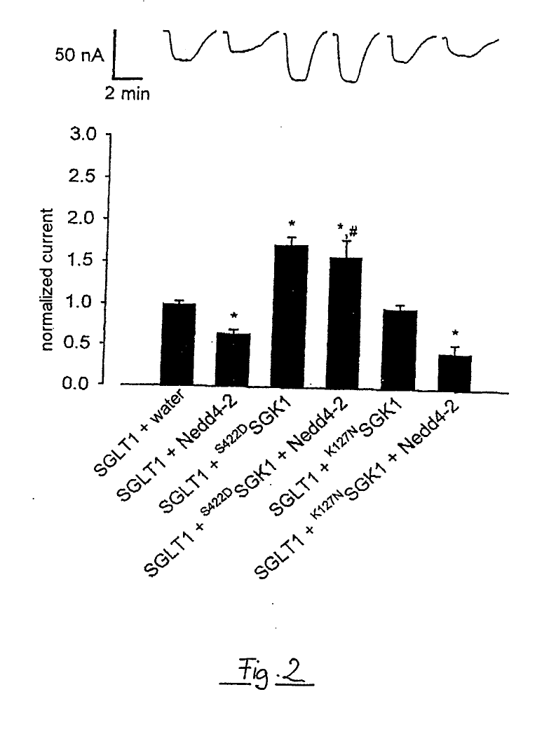

FIG. 2: shows the regulation of the Na⁺-coupled glucose transporter Sglt1 by Nedd4-2, constitutively active ^{S422D}Sgk1 and inactive ^{K127N}Sgk1.
Upper section: Originally measured curves; lower section: arithmetic means±SEM (n=8-71). Xenopus laevis oocytes were injected with sglt1, nedd4-2 and/or ^{S422D}sgk1 or ^{K127N}sgk1 cRNA. Whereas Nedd4-2 significantly downregulated the currents which are induced 20 mM glucose in oocytes which were expressing Sglt1, ^{S422D}Sgk1, but not ^{K127N}Sgk1, stimulated the currents and reversed the effect of Nedd4-2.
* indicates the differences which were significant as compared with the currents which were measured in oocytes which were expressing Sglt1 on its own.
# indicates the differences which were significant as compared with the corresponding values in oocytes which were expressing Sglt1 and Nedd4-2.

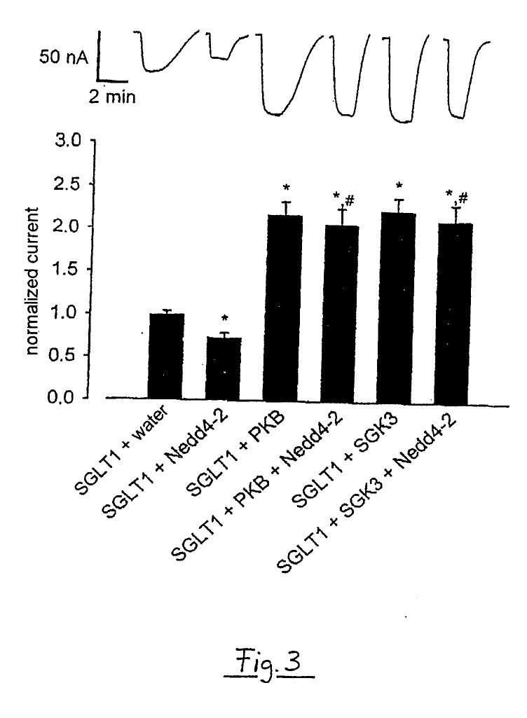

FIG. 3: shows the regulation of the Na⁺-coupled glucose transporter Sglt1 by Nedd4-2, ^{T308D,S473D}PKB and Sgk3.
Upper section: Originally measured curves; lower section: arithmetic means±SEM. Xenopus laevis oocytes were injected with sglt1, nedd4-2, ^{T308D,S473D}PKB and/or sgk3 cRNA. Nedd4-2 significantly downregulated the currents which were induced by 20 mM glucose in oocytes which were expressing Sglt1. ^{T308D,S473D}PKB and Sgk3 stimulated the currents and reversed the effect of Nedd4-2.
* indicates the differences which were significant as compared with currents which were measured in oocytes which were expressing Sglt1 on its own.
# indicates the differences which were significant as compared with the corresponding values in oocytes which were expressing Sglt1 and Nedd4-2.

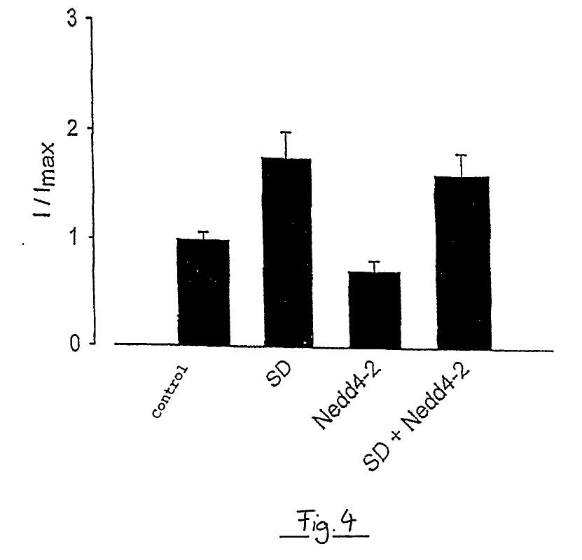

FIG. 4: shows the regulation of the Na⁺-coupled glucose transporter Sglt1 by Nedd4-2 and Sgk1. Arithmetic means±SEM (n=18). Xenopus oocytes were injected with sglt1, nedd4-2 and/or ^{S422D}Sgk1 (SD) cRNA. Whereas coexpression of Nedd4-2 reduced the currents which were induced by adding 5 mmol glucose, the currents were significantly increased by coexpressing constitutively active kinase ^{S422D}Sgk1.

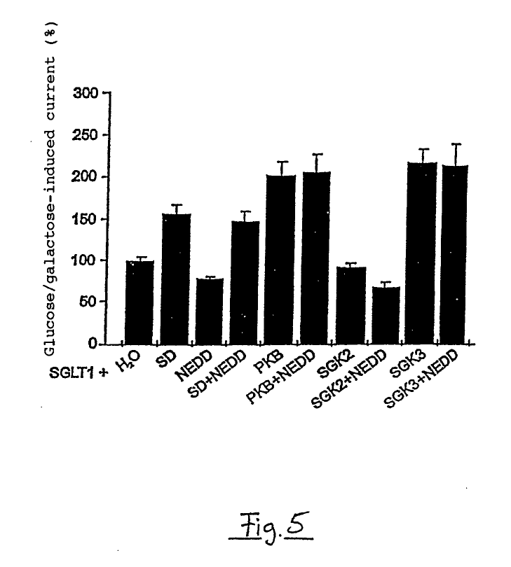

FIG. 5: shows the regulation of the Na⁺-coupled glucose transporter Sglt1 by Nedd4-2, Sgk3 and PKB. Arithmetic means±SEM (experimental procedure as in FIG. 4).

### EXAMPLE

Methods

1. Expression in Xenopus laevis Oocytes and Two-Electrode Voltage Clamp

cRNAs encoding wild-type Sgk1 [Waldegger S, Barth P, Raber G, Lang F: Cloning and characterization of a putative human serine/threonine protein kinase transcriptionally modified during anisotonic and isotonic alterations of cell volume. Proc Natl Acad Sci USA 1997; 94; 4440-4445], encoding constitutively active Sgk1 (^{S422D}Sgk1) and inactive Sgk1 (^{K127N}Sgk1) [Kobayashi T, Cohen P. Activation of serum- and glucocorticoid-regulated protein kinase by agonists that activate phosphatidylinositide 3-kinase is mediated by 3-phosphoinositide-dependent protein kinase-1 (PDK1) and PDK2. Biochem J. 1999; 339: 319-28], wild-type Sgk3 and PKB [Kobayashi T, Deak M, Morrice N, Cohen P. Characterization of the structure and regulation of two novel isoforms of serum- and glucocorticoid-induced protein kinase. Biochem J. 1999; 334: 189-97], consitutively active ^{T308D,S473D}PKB [Alessi D R, Cohen P. Mechanism of activation and function of protein kinase B. Curr. Opin Genet Dev. 1998; 8: 55-62], wild-type Nedd4-2 [Debonneville C, Flores S Y, Kamynina E, Plant P J, Tauxe C, Thomas M A, Munster C, Chraibi A, Pratt J H, Horisberger J D, Pearce D, Loffing J, Staub O. Phosphorylation of Nedd4-2 by Sgk1 regulates epithelial Na(+) channel cell surface expression. EMBO J. 2001; 20: 7052-7059] and wild-type Sglt1 [Hediger M A, Coady M J, Ikeda T S, Wright E M. Expression cloning and cDNA sequencing of the Na⁺/glucose co-transporter. Nature. 1987; 330: 379-381] were synthesized in vitro [Wagner C A, Friedrich B, Setiawan I, Lang F, Bröer S: The use of Xenopus laevis oocytes for the functional characterization of heterologously expressed membrane proteins. Cell Physiol Biochem 2000; 10: 1-12]. Dissection of the Xenopus laevis ovaries, and collection and treatment of the oocytes, have already been described in detail [Wagner C A, Friedrich B, Setiawan I, Lang F, Broer S: The use of Xenopus laevis oocytes for the functional characterization of heterologously expressed membrane proteins. Cell Physiol Biochem 2000; 10: 1-12]. The oocytes were injected with 5 ng of human sglt1, 7.5 ng of human sgk1, ^{K127N}sgk1, ^{S422D}sgk1, sgk3, PKB or ^{T308D,S473D}PKB, and/or with 5 ng of Xenopus nedd4-2. Control oocytes were injected with water. Electrophysiological experiments were carried out at room temperature for 3 days after the respective cRNAs had been injected. The currents which were induced by the extracellular administration of 20 mM or 5 mM glucose were measured using a two-electrode voltage clamp [Wagner C A, Friedrich B, Setiawan I, Lang F, Broer S: The use of Xenopus laevis oocytes for the functional characterization of heterologously expressed membrane proteins. Cell Physiol Biochem 2000; 10: 1-12] and taken as a measure of the glucose transport. The data were filtered at 10 Hz and analyzed using a MacLab Digital to Analog Converter and corresponding software (AD Instruments, Castle Hill, Australia). The control bath solution (ND 96) contained 96 mM NaCl, 2 mM KCl, 1.8 mM CaCl₂, 1 mM MgCl₂ and 5 mM HEPES, pH 7.4. All the substances were used at the stated concentrations. The final solutions were titrated with HCl or NaOH to the stated pH or pH 7.4. The flow rate of the superfusion solution was 20 ml/min and achieved complete change of solution within 10 s.

For the calculations, the data were quoted as arithmetic means±SEM. n is the number of oocytes investigated. All the experiments were carried out in at least three different groups of oocytes. Qualitatively similar data were obtained in all the repeats. The results were tested for significant differences using Student's t test. Only results giving P<0.05 were made use of as being statistically significant.

2. Studies with Twins

126 pairs of enzygotic and 70 pairs of dizygotic twins were recruited for the studies on blood pressure regulation and on cardiovascular phenotypes. The parents of the dizygotic twins were also included. All the participants were German Caucasians from different parts of Germany. Blood was removed from all the twins, and from the parents of the dizygotic twins, for the purpose of determining zygosity and for other molecular genetic studies. Each participant underwent a medical and physical examination. None of the participants had a family history of chronic medical diseases. A single nucleotide polymorphism (SNP) was identified in Exon 8 (C→T) and a second SNP was identified 551 base pairs away in the donor site (Intron 6) of Exon 7 (T→C) [Busjahn A, Aydin A, Uhlmann R et al., Serum- and glucocorticoid-regulated kinase (SGK1) gene and blood pressure. Hypertension 2002; 40:256-260)]. These two individual SNPs, i.e. Intron 6 (T→C) and Exon 8 (C→T), were analyzed.

Descriptive statistics for the two SNPs showed a recessive mode of action. The association analysis was therefore based on two group comparisons, i.e. on homozygous carriers of the variant vs. heterozygous carriers and noncarriers. The independence of the two SNPs was tested using the chi² test. The relationship between the SNPs and the phenotypes was tested by means of unidimensional ANOVA, with both polymorphisms being incorporated at the same time. This analysis related to both parts of the dizygotic twin pairs and to a randomly selected part of the enzygotic twin pairs. This test was more reliable than the t test and it was possible to take into account both polymorphisms simultaneously while including their interaction. In addition, it was also possible, in this way, to reduce the number of investigations. Since the two parts of the dizygotic twin pairs are not independent of each other, familial effects as well as age and sex were included in the ANOVA test as covariants. The significance level was set at 0.05. In the confirmation group, the association effect was tested by means of unidimensional ANOVA using both SNPs at the same time.

Results

The administration of 20 mM glucose led, in Sglt1 mRNA-injected Xenopus oocytes but not in oocytes which had been injected with water, to an inward current (I_{glc}) of 48.6±11.5 nA (n=18). By comparison, glucose treatment led, in water-injected oocytes, to a current of 1.3±0.7 nA (n=6). In Xenopus oocytes which had been injected with Sglt1 mRNA and Nedd4-2 mRNA (coexpression), the I_{glc} was significantly lowered by 49.2±6.8% (n=15). Consequently, Sglt1 is downregulated by the ubiquitin ligase Nedd4-2 (FIG. 1).

Coexpression of wild-type Sgk1 upregulated the glucose-induced current by 81.3±19.0% (n=15) and reversed the effect of Nedd4-2. In oocytes which were expressing Sglt1 together with Sgk1 and Nedd4-2, the glucose-induced current was 34.8±11.8% (n=14) higher than the value which was observed in oocytes which were expressing Sglt1 on its own (FIG. 1).

Constitutively active ^{S422D}Sgk1 had a similar effect to that of wild-type Sgk1 (FIG. 2). Coexpression of ^{S422D}Sgk1 increased the glucose-induced current by 72.4±9.1% (n=57). In this series of experiments, coexpression of Nedd4-2 lowered the current by 35.3±4.4% (n=46). This effect was reversed by additionally coexpressing ^{S422D}Sgk1. In oocytes which were coexpressing Nedd4-2 and ^{S442D}Sgk1, the current was 59.2±19.8% (n=16) higher than in oocytes which were expressing Sglt1 on its own (FIG. 2). In contrast to wild-type or constitutively active Sgk1, the inactive mutant ^{K127N}Sgk1 did not significantly alter the substrate-induced current (−2.0±5.3%, n=14) and did not reverse the effect of Nedd4-2. In oocytes which were expressing Sglt1 together with ^{K127N}Sgk1 and Nedd4-2, the glucose-induced current was 54.9±9.7% (n=8) lower than the value which was observed in oocytes which were expressing Sglt1 on its own (FIG. 2).

The effect of Sgk1 was imitated by ^{T308D,S473D}PKB (FIG. 3). In this series of experiments, coexpression of Nedd4-2 lowered the current by 26.5±5.5% (n=42). Coexpression with constitutively active ^{T308D,S473D}PKB significantly increased the glucose-induced current in oocytes which were expressing Sglt1 by 117.4±15.8% (n=31) and reversed the effect of Nedd4-2. In Xenopus oocytes which were coexpressing ^{T308D,S473D}PKB and Nedd4-2 together with Sglt1, the glucose-induced current was 106.5±18.2% (n=27) higher than the current in Xenopus oocytes which were expressing Sglt1 on its own (FIG. 3).

In a comparable manner to that of ^{T308D,S473D}PKB and Sgk1, Sgk3 stimulated the glucose-induced current and reversed the effect of Nedd4-2. The glucose-induced current was 123.6±15.0% (n=22) higher in oocytes which were expressing Sglt1 and Sgk3, and 112.4±19.4% (n=22) higher in oocytes which were expressing Sglt1, Nedd4-2 and Sgk3, than the glucose-induced current in Xenopus oocytes which were expressing Sglt1 on its own.

FIG. 4 shows that coexpression of Sglt1 and ^{S442D}Sgk1 (SD) increases the Iglc by 77±23% to 65.4±10.6 nA (n=18). In oocytes which were expressing Sglt1 together with Sgk1 and Nedd4-2, the glucose-induced current reached 60.5±9.9 nA (n=18), that is 61±21% more than the corresponding value in oocytes which were only injected with Sglt1 and 126±23% more than in oocytes which had been injected with Sglt1 and Nedd4-2 mRNA. In these experiments, the current was induced with 5 mM glucose.

In a further series of experiments, the isoforms of Sgk, i.e. Sgk2 and Sgk3, as well as protein kinase B (PKB), were tested in addition to the constitutively active ^{S422D}Sgk1 (SD). The glucose-induced current was increased by 55±12% (n=44) by coexpressing ^{S422D}Sgk1, by 117±16% (n=16) by coexpressing Sgk3, and by 101±18% (n=24) by coexpressing PKB, while Sgk2 had no statistically significant effect. While coexpressing Nedd4-2 lowered glucose transport by 23±4% (n=79), it did not prevent stimulation by the additional coexpression of ^{S122D}Sgk1 (+48±11%, n=48), of Sgk3 (+114±26%, n=16) and of PKB (+107±20%, n=24). Once again, Sgk2 had no significant effect.

In order to investigate the functional relevance of Sgk1 in the regulation of Sglt1 and bodyweight, the body mass index of twins possessing polymorphisms of the Sgk1 gene was correlated. The average body mass of twins which were carrying the polymorphism E8CC/CT;I6CC amounted to 26.7±1.4 kg/M² (n=13). This value is significantly higher (P<0.008) than the corresponding average values (23.3±0.2 kg/m², n=263) for the twins as a whole.

Taken overall, the experiments demonstrate that Sgk1, Sgk3 and PKB have a strong stimulatory effect on Sglt1. The increase in Sglt1 activity accelerates the intestinal absorption of glucose such that the concentration of glucose in the plasma increases more rapidly. This increases the release of insulin and thereby stimulates the deposition of lipid in adipose tissue. On the other hand, inhibitors of Sglt1 counter-act corpulence.

The studies with twins demonstrate that the same polymorphism which is associated with elevated blood pressure is also connected to a higher body mass index.

1-52. (canceled)

53. A method for diagnosing diseases which are associated with disturbed glucose transport wherein at least one substance for detecting the expression and/or function of activated and/or inactive Sgk, in particular Sgk1 and/or Sgk3, and/or PKB, and/or Nedd, in particular Nedd4-2, is used.

54. The method of claim 53, characterized in that the substance is at least one substance selected from the group of antibodies and nucleotides.

55. The method of claim 53, characterized in that use is made of antibodies which are directed against phosphorylated and/or unphosphorylated sequences in Sgk, in particular Sgk1 and/or Sgk3, PKB and/or Nedd, in particular Nedd4-2.

56. The method of claim 55, characterized in that use is made of antibodies which are directed against at least one phosphorylated and/or unphosphorylated kinase consensus sequence, in particular an Sgk1 consensus sequence, in a Nedd protein, in particular in the Nedd4-2 protein.

57. The method of claim 53, characterized in that at least one mutation, in particular an inactivating mutation, is detected in Nedd, in particular in nedd4-2, in DNA, RNA and/or a Nedd protein from a biological sample, in particular a sample from a patient, with the mutation preferably being present in a segment of nedd which encodes an Sgk1 consensus sequence in the Nedd protein.

58. The method of claim 57, characterized in that the mutation is ^{S338D}Nedd4-2 and/or ^{S444D}Nedd4-2.

59. The method of claim 53, characterized in that at least one mutation, in particular an activating mutation, is detected in sgk, in particular in sgk1 and/or sgk3, and/or a gene for PKB, in DNA, RNA and/or an Sgk protein and/or PKB protein from a biological sample, in particular a sample from a patient.

60. The method of claim 59, characterized in that the mutation is ^{S422D}Sgk1 and/or ^{T308D,S473D}PKB.

61. The method of claim 53, characterized in that the diseases are the metabolic syndrome, in particular obesity.

62. A method for diagnosing predispositions to obesity, characterized in that at least one polymorphism is detected in sgk, in particular sgk1 and/or sgk3, a gene for PKB, nedd, in particular nedd4-2, and/or sglt, in particular sglt1.

63. The method of claim 62, characterized in that the polymorphism is a single nucleotide polymorphism (SNP).

64. The method of claim 62, characterized in that the polymorphism is E8CC/CT;I6CC in Sgk1.

65. A method for exerting an effect on glucose transport, in particular intestinal and/or renal glucose transport in which at least one active compound is used.

66. The method of claim 65, characterized in that the active compound exerts an effect on at least one Sgk, in particular Sgk1 and/or Sgk3, and/or PKB, and/or an effect on at least one Nedd, in particular Nedd4-2.

67. The method of claim 65, characterized in that the active compound is directed against an Sgk, in particular Sgk1 and/or Sgk3, and/or PKB and/or a Nedd, in particular Nedd4-2.

68. The method of claim 65, characterized in that the active compound is directed against activators, inhibitors, regulators and/or biological precursors of an Sgk, in particular of Sgk1 and/or Sgk3, and/or PKB and/or a Nedd, in particular Nedd4-2.

69. The method of claim 65, characterized in that the active compound is a polynucleotide which preferably encodes a peptide, in particular a polypeptide.

70. The method of claim 65, characterized in that the active compound is a peptide, preferably a polypeptide.

71. The method of claim 70, characterized in that the peptide exerts an effect on the expression and/or function of an Sgk, in particular Sgk1 and/or Sgk3, and/or PKB and/or a Nedd, in particular Nedd4-2.

72. The method of claim 65, characterized in that the active compound is a “small molecular compound”, preferably a “small molecular compound” having a molecular weight (MW) of <1000.

73. The method of claim 65, characterized in that the active compound inhibits at least one Sgk, in particular Sgk1 and/or Sgk3, and/or PKB, and/or stimulates at least one Nedd, in particular Nedd4-2, in particular for the purpose of preventing or treating diseases which are connected with disturbed glucose absorption.

74. The method of claim 65, characterized in that the active compound is at least one kinase inhibitor, preferably staurosporine and/or chelerythrine, or one of their analogs, and/or at least one ligase activator.

75. A method for treating diseases which are connected with disturbed glucose transport in which at least one active compound for exerting an effect on, in particular inhibiting, at least one Sgk and/or PKB, and/or for exerting an effect on, in particular stimulating, at least one Nedd, is used.

76. The method of claim 73, characterized in that the diseases are the metabolic syndrome, in particular obesity.

77. The method of claim 65, characterized in that the active compound stimulates at least one Sgk, in particular Sgk1 and/or Sgk3, and/or PKB, and/or inhibits at least one Nedd, in particular Nedd4-2, for the purpose of increasing glucose transport, in particular for increasing the bodyweight of animals.

78. The method of claim 77, characterized in that the active compound is at least one Sgk activator and/or PKB activator, in particular a growth factor, preferably IGF1, and/or insulin.

79. The method of claim 77, characterized in that the active compound is at least one stimulant of the transcription of sgk1 and/or sgk3 and/or a gene for PKB, preferably at least one glucocorticoid, mineral corticoid, gonadotropin and/or cytokine, in particular TGFβ.

80. A diagnostic kit which comprises at least one substance for detecting the expression and/or function of activated and/or inactive Sgk, in particular Sgk1 and/or Sgk3, and/or PKB and/or Nedd, in particular Nedd4-2, for diagnosing diseases which are associated with disturbed glucose transport.

81. The diagnostic kit of claim 80, characterized in that the diseases are the metabolic syndrome, in particular obesity.

82. An antibody, characterized in that it is directed against at least one phosphorylated kinase consensus sequence, in particular an Sgk1 consensus sequence, in a Nedd protein, in particular in the Nedd4-2 protein.

83. An antibody, characterized in that it is directed against at least one unphosphorylated kinase consensus sequence, in particular an Sgk1 consensus sequence, in a Nedd protein, in particular in the Nedd4-2 protein.

84. An antibody, characterized in that it is directed against at least one mutated kinase consensus sequence, in particular an Sgk1 consensus sequence, in a Nedd protein, in particular in the Nedd4-2 protein.

85. The antibody of claim 84, characterized in that the Nedd protein with a mutated kinase consensus sequence is ^{S338N}edd4-2 and/or ^{S444D}Nedd4-2.

86. A composition, in particular a pharmaceutical composition, comprising an effective quantity of at least one active compound which exerts an effect on glucose transport, in particular intestinal and/or renal glucose transport, and, where appropriate, a pharmaceutically acceptable excipient.

87. The composition of claim 86, characterized in that the active compound exerts an effect on at least one Sgk and/or PKB and/or at least one Nedd.

88. The composition of claim 86, characterized in that the active compound exerts an effect on activators, inhibitors, regulators and/or biological precursors of an Sgk, in particular of Sgk1 and/or Sgk3, and/or PKB and/or a Nedd, in particular Nedd4-2.

89. The composition of claim 86, characterized in that the active compound is a polynucleotide which preferably encodes a peptide, in particular a polypeptide.

90. The composition of claim 86, characterized in that the active compound is a peptide, preferably a polypeptide.

91. The composition of claim 90, characterized in that the peptide exerts an effect on the expression and/or function of an Sgk, in particular Sgk1 and/or Sgk3, and/or PKB and/or a Nedd, in particular Nedd4-2.

92. The composition of claim 86, characterized in that the active compound is a “small molecular compound”, preferably a “small molecular compound” having a molecular weight (MW) of <1000.

93. The composition of claim 86, characterized in that the active compound inhibits at least one Sgk and/or PKB and/or stimulates at least one Nedd.

94. The composition of claim 86, characterized in that the active compound is at least one kinase inhibitor, preferably staurosporine and/or chelerythrine or one of their analogs, and/or at least one ligase activator.

95. The composition of claim 86, characterized in that the active compound stimulates at least one Sgk and/or PKB and/or inhibits at least one Nedd.

96. The composition of claim 95, characterized in that the active compound is at least one Sgk activator and/or PKB activator, in particular a growth factor, preferably IGF1, and/or insulin.

97. The composition of claim 95, characterized in that the active compound is at least one stimulant of the transcription of sgk1 and/or sgk3 and/or a gene for PKB, preferably at least one glucocorticoid, mineral corticoid, gonadotropin and/or cytokine, in particular TGFβ.

98. A method for producing transgenic animals, excluding humans, which exhibit an increase in lipid deposition in adipose tissue, characterized in that the expression and/or function of Sglt, in particular Sglt1, is increased.

99. The method of claim 98, characterized in that Sglt, in particular Sglt1, is overexpressed.

100. The method of claim 98, characterized in that the expression and/or function of at least one Sgk, in particular Sgk1 and/or Sgk3, and/or PKB, is increased.

101. The method of in claim 100, characterized in that at least one sgk, in particular sgk1 and/or sgk3, and/or at least one gene for PKB, is overexpressed.

102. The method of claim 100, characterized in that use is made of at least one activating mutation of sgk, in particular of sgk1 and/or sgk3, and/or of a gene for PKB, in particular ^{S422D}sgk1 and/or ^{T308D,S473D}PKB.

103. The method of claim 98, characterized in that the expression and/or function of at least one Nedd, in particular Nedd4-2, is decreased.

104. The method of in claim 103, characterized in that use is made of at least one inactivating mutation of nedd, in particular of nedd4-2, in particular ^{S338D}nedd4-2 and/or ^{S444D}nedd4-2.
